(19) United States
(12) Patent Application Publication
(10) Pub. No.: US 2014/0256813 A1
(43) Pub. Date: Sep. 11, 2014

(54) METHODS FOR THE TREATMENT OF BRAIN INJURY USING OMEGA-3 FATTY ACIDS

(71) Applicant: Nordic Naturals, Inc., Watsonville, CA (US)

(72) Inventor: Joar A. Opheim, Aptos, CA (US)

(73) Assignee: Nordic Naturals, Inc., Watsonville, CA (US)

(21) Appl. No.: 14/201,353

(22) Filed: Mar. 7, 2014

Related U.S. Application Data
(60) Provisional application No. 61/774,847, filed on Mar. 8, 2013.

(51) Int. Cl. A61K 31/202

(52) U.S. Cl. CPC A61K 31/202
USPC 514/560

(57) ABSTRACT

Methods of treating, managing or preventing brain injury are disclosed. Specific methods encompass the administration of EPA and DHA triglycerides for the treatment of brain injuries.

### CROSS REFERENCE TO RELATED APPLICATIONS

The present application claims the benefit of priority of U.S. Provisional Application No. 61/774,847, filed Mar. 8, 2013, the content of which is hereby incorporated by reference in its entirety.

### FIELD

Provided herein are methods for the treatment of brain injury using omega-3 fatty acids. Also provided herein are methods for the treatment of a brain injury using omega-3 fatty acids, wherein the omega-3 fatty acid comprises EPA and DHA triglycerides.

### BACKGROUND

Brain injuries may occur by trauma to the whole body, including head and/or brain, or by other mechanisms such as stroke, heart attack, drowning, gas poisoning (e.g., carbon monoxide) or other occurance which results in a loss of oxygen to the brain.

Traumatic brain injury (TBI) has long been recognized as a leading cause of traumatic death and disability. Tremendous advances in surgical and intensive care unit management of the primary injury, including maintaining adequate oxygenation, controlling intracranial pressure and ensuring proper cerebral perfusion, have resulted in reduced mortality. See J. Neurotrauma, 2007, 24 (Supp. 1): S1-106. However, the secondary injury phase of TBI is a prolonged pathogenic process characterized by neuroinflammation, excitatory amino acids, free radicals, and ionimbalance. See Ling et al., Neurol. Clin., 2008, 26(2): 409-426. There are no approved therapies to directly address these underlying processes.

EPA and DHA are omega-3 polyunsaturated fatty acids (n-3FA). EPA is 5,8,11,14,17-eicosapentaenoic acid (eicosapentaenoic acid or “EPA,” (20:5 (n-3)) and DHA is 4,7,10,13,16,19-docosahexaenoic acid (docosahexaenoic acid or “DHA,” 22:6 (n-3)). EPA and DHA can be found in nature (e.g., in fish oils), and have been used in a variety of dietary/therapeutic compositions.

The term “omega-3 polyunsaturated fatty acid(s)” refers to the fact that EPA and DHA have a carbon-carbon double bond in the n-3 position (i.e., the third bond from the methyl end of the molecule). EPA and DHA frequently have all of their carbon-carbon double bonds in the cis-configuration.

It is well-recognized that omega-3 fatty acids (n-3FA) are important for proper neurodevelopment and function. See Forsyth et al., Lancet, Aug. 29, 1998, 352(9129): 688-691. However, average Western dietary intakes result in a deficiency of n-3FA and an over-dominant intake of proinflammatory omega-6s (n-6FA). The ratio of n-3:n-6FA in the Western diet can be as low as 1:50. Such imbalance is reflected directly in the composition of neuron membrane phospholipids favoring inflammatory processes. See Bistrian, B. R., J. Parenteral and Enteral Nutrition, 2003, 27(3): 168-175.

Arachidonic Acid, the primary n-6FA in the brain, is metabolized by cyclooxygenase (COX) and lipoxygenase (LOX) enzymes to pro-inflammatory eicosanoids that enhance vascular peiineability, increase local blood flow, increase infiltration of leukocytes, and enhance production of proinflammatory cytokines. See Calder, P. C., Braz. J. Med. Biol. Res., 2003, 36(4): 433-446. n-3FA attenuate release of these proinflammatory cytokines, decrease COX activity, inhibit formation of proinflammatory eicosanoids and cytokines, and promote levels of anti-inflammatory decosanoids.

The n-3FA docosahexaenoic acid (DHA), α-linolenic acid (LNA), and eicosapentaenoic acid (EPA) have been hypothesized to have a positive influence on traumatic brain and spinal cord injury. See Michel-Titus, A. T., Clin. Lipidol., 2009, 4(3): 343-353. Laboratory animal research shows that n-3FA may help improve clinical outcomes when administered prior to or following TBI or spinal cord injury (SCl). See Mills et al., J. Neurotrama, 2011, 114(1): 77-84; Bailes et al., J. Neurotrama, 2010, 27(1): 1617-1624; Huang et al., Brain, 2007, 130: 3004-3019. In such studies, n-3FA, as well as DHA alone, significantly reduce the number of injured axons, and results in retention of neuromotor function. Treatment with n-3FA represents a potentially promising therapeutic approach for neurotrauma which would be easy to translate to the emergency patient-care arena considering the well-documented safety and tolerability of these compounds.

### SUMMARY

Provided herein are methods of treating a brain injury in a patient, comprising oral administration of a composition comprising an omega-3 fatty acid mixture to a patient having a brain injury, wherein the omega-3 fatty acid mixture comprises at least about 70% by weight of a combination of (a) EPA glyceride, and (b) DHA glyceride, in a weight ratio of a:b of from about 1.5:1 to about 1.3:1.

In some embodiments, the omega-3 fatty acid mixture comprises at least about 80% by weight of a combination of (a) EPA glyceride, and (b) DHA glyceride, in a weight ratio of a:b of from about 1.5:1 to about 1.3:1.

In some embodiments, the omega-3 fatty acid mixture comprises at least about 85% by weight of a combination of (a) EPA glyceride, and (b) DHA glyceride, in a weight ratio of a:b of from about 1.5:1 to about 1.3:1.

In some embodiments, the ratio of a:b is from about 1.5:1 to about 1.4:1. In some embodiments, the ratio of a:b is about 1.4:1. In some embodiments, the ratio of a:b is about 1.5:1.

In some embodiments, the triglyceride content of the EPA glyceride is about 80%, 85%, 90%, 95%, 98% or 99% by weight.

In some embodiments, the triglyceride content of the DHA glyceride is about 80%, 85%, 90%, 95%, 98% or 99% by weight.

In some embodiments, about 15,000 mg to about 25,000 mg of a combination of EPA glyceride and DHA glyceride is administered daily. In some embodiments, about 15,000 mg to about 20,000 mg of a combination of EPA glyceride and DHA glyceride is administered daily. In some embodiments, about 15,000 mg to about 16,000 mg of a combination of EPA glyceride and DHA glyceride is administered daily.

In some embodiments, the composition is administered twice daily. In some embodiments, about 7,500 mg to about 10,000 mg of a combination of EPA glyceride and DHA glyceride is administered twice daily. In some embodiments, about 7,500 mg to about 8,000 mg of a combination of EPA glyceride and DHA glyceride is administered twice daily.

In some embodiments, the composition is a liquid formulation. In other embodiments, the composition is a capsule formulation.

In some embodiments, the brain injury is a traumatic brain injury. In some embodiments, the method further comprises the amelioriation of damage associated with the traumatic brain injury.

In some embodiments, the method provided herein prevents damage to the brain associated with a traumatic brain injury by reducing secondary brain trauma in the patient post-acute brain injury.

In some embodiments, the brain injury is an anoxic brain injury.

The methods described herein may be also be administered in combination with other known methods of treating brain injuries.

### DETAILED DESCRIPTION

Provided herein are methods of treating a brain injury in a patient, comprising oral administration of a composition comprising an omega-3 fatty acid mixture to a patient having a brain injury.

In some embodiments, the omega-3 fatty acid mixture comprises at least about 70% by weight of a combination of (a) EPA, pharmaceutically acceptable derivatives of EPA or mixtures thereof and (b) DHA, pharmaceutically acceptable derivatives of DHA or mixtures thereof in a weight ratio of a:b of from about 1.5:1 to about 1.3:1. In some embodiments, the derivatives of EPA and derivatives of DHA are glycerides. In some embodiments, the derivatives of EPA and derivatives of DHA are triglycerides.

In some embodiments, provided herein are methods of treating a brain injury in a patient, comprising oral administration of a composition comprising an omega-3 fatty acid mixture to a patient having a brain injury, wherein the omega-3 fatty acid mixture comprises at least about 70% by weight of a combination of (a) EPA glyceride, and (b) DHA glyceride, in a weight ratio of a:b of from about 1.5:1 to about 1.3:1.

In some embodiments, the omega-3 fatty acid mixture comprises at least about 80% by weight of a combination of (a) EPA glyceride, and (b) DHA glyceride, in a weight ratio of a:b of from about 1.5:1 to about 1.3:1.

In some embodiments, the omega-3 fatty acid mixture comprises at least about 85% by weight of a combination of (a) EPA glyceride, and (b) DHA glyceride, in a weight ratio of a:b of from about 1.5:1 to about 1.3:1.

In some embodiments, the omega-3 fatty acid mixture comprises at least about 90% by weight of a combination of (a) EPA glyceride, and (b) DHA glyceride, in a weight ratio of a:b of from about 1.5:1 to about 1.3:1.

In some embodiments, the omega-3 fatty acid mixture comprises at least about 95% by weight of a combination of (a) EPA glyceride, and (b) DHA glyceride, in a weight ratio of a:b of from about 1.5:1 to about 1.3:1.

In some embodiments, the omega-3 fatty acid mixture comprises at least about 98% by weight of a combination of (a) EPA glyceride, and (b) DHA glyceride, in a weight ratio of a:b of from about 1.5:1 to about 1.3:1.

In some embodiments, the omega-3 fatty acid mixture comprises at least about 99% by weight of a combination of (a) EPA glyceride, and (b) DHA glyceride, in a weight ratio of a:b of from about 1.5:1 to about 1.3:1.

In some embodiments, provided herein are methods of treating a brain injury in a patient, comprising oral administration of a composition comprising an omega-3 fatty acid mixture to a patient having a brain injury, wherein the omega-3 fatty acid mixture comprises at least about 70% by weight of a combination of (a) EPA glyceride, and (b) DHA glyceride, in a weight ratio of a:b of from about 1.5:1 to about 1.4:1.

In some embodiments, the omega-3 fatty acid mixture comprises at least about 80% by weight of a combination of (a) EPA glyceride, and (b) DHA glyceride, in a weight ratio of a:b of from about 1.5:1 to about 1.4:1.

In some embodiments, the omega-3 fatty acid mixture comprises at least about 85% by weight of a combination of (a) EPA glyceride, and (b) DHA glyceride, in a weight ratio of a:b of from about 1.5:1 to about 1.4:1.

In some embodiments, the omega-3 fatty acid mixture comprises at least about 90% by weight of a combination of (a) EPA glyceride, and (b) DHA glyceride, in a weight ratio of a:b of from about 1.5:1 to about 1.4:1.

In some embodiments, the omega-3 fatty acid mixture comprises at least about 95% by weight of a combination of (a) EPA glyceride, and (b) DHA glyceride, in a weight ratio of a:b of from about 1.5:1 to about 1.4:1.

In some embodiments, the omega-3 fatty acid mixture comprises at least about 98% by weight of a combination of (a) EPA glyceride, and (b) DHA glyceride, in a weight ratio of a:b of from about 1.5:1 to about 1.4:1.

In some embodiments, the omega-3 fatty acid mixture comprises at least about 99% by weight of a combination of (a) EPA glyceride, and (b) DHA glyceride, in a weight ratio of a:b of from about 1.5:1 to about 1.4:1.

In some embodiments, the ratio of a:b is from about 1.6:1 to about 1.4:1. In some embodiments, the ratio of a:b is about 1.4:1. In some embodiments, the ratio of a:b is about 1.45:1. In some embodiments, the ratio of a:b is about 1.5:1.

In some embodiments, the triglyceride content of the EPA glyceride is about 80%, 81%, 82%, 83%, 84%, 85%, 86%, 87%, 88%, 89%, 90%, 91%, 92%, 93%, 94%, 95%, 96%, 97%, 98% or 99% by weight. In some embodiments, the triglyceride content of the EPA glyceride is about 90% by weight.

In some embodiments, the triglyceride content of the DHA glyceride is about 80%, 81%, 82%, 83%, 84%, 85%, 86%, 87%, 88%, 89%, 90%, 91%, 92%, 93%, 94%, 95%, 96%, 97%, 98% or 99% by weight. In some embodiments, the triglyceride content of the DHA glyceride is about 90% by weight.

In some embodiments, 15,000 mg to about 25,000 mg of a combination of EPA glyceride and DHA glyceride is administered daily. In some embodiments, about 15,000 mg to about 20,000 mg of a combination of EPA glyceride and DHA glyceride is administered daily. In some embodiments, about 15,000 mg to about 16,000 mg of a combination of EPA glyceride and DHA glyceride is administered daily. In some embodiments, about 15,000 mg of a combination of EPA glyceride and DHA glyceride is administered daily. In some embodiments, about 16,000 mg of a combination of EPA glyceride and DHA glyceride is administered daily.

In some embodiments, the composition is administered twice daily. In some embodiments, about 7,500 mg to about 12,500 mg of a combination of EPA glyceride and DHA glyceride is administered twice daily. In some embodiments, about 7,500 mg to about 10,000 mg of a combination of EPA glyceride and DHA glyceride is administered twice daily. In some embodiments, about 7,500 mg to about 8,000 mg of a combination of EPA glyceride and DHA glyceride is administered twice daily. In some embodiments, about 7,500 mg of a combination of EPA glyceride and DHA glyceride is administered twice daily. In some embodiments, about 8,000 mg of a combination of EPA glyceride and DHA glyceride is administered twice daily.

In some embodiments, the composition is a liquid founulation. In other embodiments, the composition is a capsule formulation. In some embodiments, the composition further comprises a flavoring agent. In some embodiments the flavoring agent is rosemary extract or lemon oil. In some embodiments, the composition further comprisies vitamin E (i.e., d-alpha tocopherol).

In some embodiments, the brain injury is a traumatic brain injury. In some embodiments, the method further comprises the amelioriation of damage associated with the traumatic brain injury.

In some embodiments, the method provided herein prevents damage to the brain associated with a traumatic brain injury by reducing secondary brain trauma in the patient post-acute brain injury.

In some embodiments, the brain injury is an anoxic brain injury.

In one embodiment, provided herein is a method of treating a brain injury in a patient, comprising oral administration of a composition comprising an omega-3 fatty acid mixture to a patient having a brain injury, wherein the omega-3 fatty acid mixture comprises at least about 80% by weight of a combination of (a) EPA triglyceride, and (b) DHA triglyceride, in a weight ratio of a:b of about 1.5:1.

In one embodiment, provided herein is a method of treating a brain injury in a patient, comprising oral administration of a composition comprising an omega-3 fatty acid mixture to a patient having a brain injury, wherein the omega-3 fatty acid mixture comprises at least about 80% by weight of a combination of (a) EPA triglyceride, and (b) DHA triglyceride, in a weight ratio of a:b of about 1.4:1.

In one embodiment, provided herein is a method of treating a traumatic brain injury in a patient, comprising oral administration of a composition comprising an omega-3 fatty acid mixture to a patient having a traumatic brain injury, wherein the omega-3 fatty acid mixture comprises at least about 80% by weight of a combination of (a) EPA triglyceride, and (b) DHA triglyceride, in a weight ratio of a:b of about 1.5:1.

In one embodiment, provided herein is a method of treating a traumatic brain injury in a patient, comprising oral administration of a composition comprising an omega-3 fatty acid mixture to a patient having a traumatic brain injury, wherein the omega-3 fatty acid mixture comprises at least about 80% by weight of a combination of (a) EPA triglyceride, and (b) DHA triglyceride, in a weight ratio of a:b of about 1.4:1.

In one embodiment, provided herein is a method of treating an anoxic brain injury in a patient, comprising oral administration of a composition comprising an omega-3 fatty acid mixture to a patient having an anoxic injury, wherein the omega-3 fatty acid mixture comprises at least about 80% by weight of a combination of (a) EPA triglyceride, and (b) DHA triglyceride, in a weight ratio of a:b of about 1.5:1.

In one embodiment, provided herein is a method of treating an anoxic brain injury in a patient, comprising oral administration of a composition comprising an omega-3 fatty acid mixture to a patient having an anoxic brain injury, wherein the omega-3 fatty acid mixture comprises at least about 80% by weight of a combination of (a) EPA triglyceride, and (b) DHA triglyceride, in a weight ratio of a:b of about 1.4:1.

The methods described herein may be also be administered in combination with other known methods of treating brain injuries.

### DEFINITIONS

As used herein, the term “patient” refers to a mammal, particularly a human. In some embodiments, the patient is a female. In further embodiments, the patient is a male. In further embodiments, the patient is a child.

As used herein, and unless otherwise specified, the teims “treat,” “treating” and “treatment” contemplate an action that occurs while a patient is suffering from the specified disease or disorder, which reduces the severity or symptoms of the disease or disorder, or retards or slows the progression or symptoms of the disease or disorder.

As used herein, unless otherwise specified, the terms “prevent,” “preventing” and “prevention” contemplate an action that occurs before a patient begins to suffer from the specified disease or disorder, which inhibits or reduces the severity or symptoms of the disease or disorder. In some embodiments, prevention refers to the reduction of further damage to the brain associated with traumatic brain injury, i.e., after an acute, kinetic head injury.

As used herein, “brain injury” refers to a traumatic brain injury and/or an anoxic brain injury.

As used herein, “traumatic brain injury” (TBI) refers to a whole body injury, including a head injury and/or a brain injury, wherein a trauma causes damage to the brain. The damage may be confined to one area of the brain or involve more than one area of the brain. Traumatic brain injury is believed to involve primary and secondary injury phases. The primary injury is represented by the moment of impact. Secondary injury is characterized by neuroinflammation, excitatory amino acids, free radicals, and ionimbalance. Symptoms may also include one or more of hypotension, hypoxia, edema, and potentially additional abnormalities in glucose utilization, cellular metabolism, membrane fluidity, synaptic function, and structural integrity of the brain.

Clinically, traumatic brain injury can be rated as mild, moderate or severe based on TBI variables that include duration of loss of consciousness (LOC), Glasgow Coma Score (GCS) and post traumatic stress amnesia. See Levin et al., J. Nervous Mental Dis., 1979, 167: 675-84; Holm et al., J. Rehabil. Med., 2005, 37:137-41.

As used herein, “secondary brain trauma” refers to damage to the brain of a patient post-acute brain injury, i.e., during the secondary injury phase of a TBI.

As used herein, “anoxic brain injury” refers to an injury to the brain as a result of a lack of oxygen to the brain, e.g., following a stroke, heart attack, drowning, carbon monoxide or other gas poisoning.

As used herein, the term “about” means that the value or amount to which it refers can vary by ±5%, ±2%, or ±1%. Percent variability can be measured in weight percent, volume percent, or mole percent.

As used herein, the terms “EPA derivative(s)” and “DHA derivative(s)” refer to EPA and DHA that have been reacted with another compound or otherwise modified so that the EPA and DHA no longer contains a free carboxylic acid. Examples of EPA and DHA derivatives include salts, esters (such as alkyl esters including, but not limited to, methyl and ethyl esters) and glycerides. The EPA and DHA can also be one or more of the fatty acid moieties in a phospholipid molecule. Since the derivatives are intended to be administered to a subject, they should be pharmaceutically acceptable. As used herein, the term “pharmaceutically acceptable” means that the material to which it refers is not harmful to the subject.

As used herein, the term “glyceride” means a glycerol molecule (i.e., OHCH₂CHOHCH₂OH) in which one, two or all three of the hydroxyls have been esterified with a carboxylic acid, e.g., an omega-3 polyunsaturated fatty acid. Thus, “triglyceride” refers to glycerides in which all three hydroxyls on the glycerol have been esterified with (the same or different) carboxylic acids. “Diglyceride” refers to glycerides in which only two of the hydroxyls on the glycerol have been esterified with (the same or different) carboxylic acids. “Monoglyceride” refers to glycerides in which only one hydroxyl on the glycerol has been esterified with a carboxylic acid.

As used herein, “EPA glyceride” refers to a glycerol molecule in which one, two or all three of the hydroxyls have been esterified with EPA. In some embodiments, EPA glyceride refers to a mixture of glycerol molecules wherein at least one of each of the hydroxyls of each glycerol molecule has been esterified with EPA.

As used herein, “DHA glyceride” refers to a glycerol molecule in which one, two or all three of the hydroxyls have been esterified with DHA. In some embodiments, DHA glyceride refers to a mixture of glycerol molecules wherein at least one of each of the hydroxyls of each glycerol molecule has been esterified with DHA.

Omega-3 fatty acids are found in nature in the triglyceride form (a glycerol with three fatty acids attached). The natural triglyceride form as found in raw fish oil cannot be readily separated as it occurs into purified EPA/DHA mixtures by ordinary means such as distillation or crystallization, because the fatty acids are non-uniformly distributed among the triglyceride molecules. There are very few, if any, single triglyceride molecules which are composed of either three EPAs or three DHAs. Typically, there is a DHA, an EPA, and another fatty acid in a triglyceride molecule. So in order to purify fatty acids to increase the proportion of EPA, DHA, or the total fraction of omega-3's, it is necessary to hydrolyze the triglycerides to remove at least some fatty acids from the glycerol.

The triglycerides may be converted by any method known to one skilled in the art without limitation. For example, the triglycerides may be converted by lipase-catalyzed esterification or lipase catalyzed acidolysis with ethyl or lauryl alcohol, which can selectively leave the highest amount of EPA and DHA bonded to glycerols and remove other components, leaving EPA and/or DHA as mono- or di-glycerides. The mono- and di-glycerides can then be separated into fractions with different EPA/DHA ratios, by methods familiar to those skilled in the art such as multiple stage vacuum distillation and/or fractional crystallization in urea. Advantageously, the purified EPA and DHA esters, after concentration, can be reattached to glycerol molecules using enzymatic reacylation to recreate glycerides which are otherwise identical to the original natural triglycerides, except that they are more concentrated in EPA and DHA combined, and they may also have a different ratio of EPA:DHA than the original fish oil. In some embodiments, at least 60% of the omega-3 fatty acids, and preferably 70% or more are converted to the triglyceride form in the reacylation process. The process may be successively repeated with addition of additional catalyst and/or enzyme and additional EPA and DHA until the desired specification proportions are met. About 60% of triglycerides can be made in the first pass of reacylation, with most of the remainder of the product being mono- and di-glycerides.

Polyunsaturated fatty acid triglycerides may be prepared using the following method.

1. Removal of Free Fatty Acids

Raw fish oil in the natural triglyceride molecular form preferably from anchovies and sardines which contain about 18% EPA and 12% DHA is heated to 60° C. to decrease viscosity. Sodium oxide is added to bind with free fatty acids in the oil. The mixture is moved to a separator where sodium oxide bound to free fatty acids (soap) floats to the top and is removed.

The oil is then moved to a second separator where warm water is preferably added to help remove traces of sodium oxide, as sodium oxide partitions to water, yet does not interact with the fish oil.

Citric acid may then be added to support splitting the oil from the combination of water and sodium oxide. The oil is then cooled to 30° C. to protect it from oxidation.

2. Stripping and Purification

Oil is moved to a separate stripping tank, and heated to 200° C. Ethyl esters can be added to support the removal of impurities, which bind to ethyl esters. Impurities such as dioxins, heavy metals, polychlorinated biphenyls (PCBs), fire retardants, furans and others evaporate and are drawn to the middle of the tank where a refrigerating element cools them down and drain them. The added esters are also removed with the impurities.

3. Esterification

The oil is moved to an esterification tank. Ethanol and sodium metal are added. Sodium metal is a catalyst for breaking off fatty acid strands from the glycerol backbone of the triglyceride fatty acid molecule, the free fatty acids then combined with ethanol to form ethyl esters. Water can be added to bind to sodium metal, where the combination of water and sodium metal can be removed.

4. Molecular Distillation

The oil is then moved to a distiller where it is heated to about 120° C. under vacuum. Mono esters and shorter carbon chain molecules move to the middle where they are cooled and drained, leaving longer carbon chains remaining as a concentrate. The process typically increases the key fatty acids by 100% during the first distillation; typically between 30-50% during the second distillation. The process can be repeated, although preferably the process is ideally only repeated once, as when oils undergo heat it can produce oxidation and degradation of the fatty acids in general. Oil waste is also increasing with repeated distillation, making the process less economical.

Oils having higher EPA content can be produced by repeating the molecular distillation step to separate EPA from other fatty acids, including separation from DHA.

5. Reesterification (Reacylation)

The oil is then moved to a reesterification tank where the ethyl ester molecules are reconverted to the triglyceride form, which is the natural form of that fatty acid molecule. 98% of fats ingested by humans are in this natural triglyceride form.

The esterification process takes place under low vacuum at about 80° C. Glycerol is added to form the backbone of the glyceride molecules. Nitrogen can be added from the bottom of the tank to cause oil movement. Lipase enzymes are added as catalysts to facilitate the fatty acids binding to glycerol. The vacuum in the distillation tank removes the ethanol which was previously bound to the fatty acids. The enzymes used are lipases produced from bacteria or yeast. Perhaps the most effective enzymes are Candidan Antarctica lipase, and Chromobacterium Viscosum Lipase; other enzymes that can be used effectively are Psuedomonas, Mucor miehei, and Candida Cylindracea as well as other enzymes may also be used.

The reesterification process typically takes 24 hours, at which point the triglycerides typically reaches 60-65%, the remaining glycerides being diglycerides and monoglycerides. Around 3% of the fish oil will remain as ethyl esters, which can be removed together with the ethanol. Adding additional enzymes and/or continuing the enzymatic process can produce triglyceride molecule concentration of up to 99%. The 60-65% level is probably optimum from an economic point of view.

6. Winterization

The oil in triglyceride form is then moved to a cooling tank at 0° C., where saturated fats, in particular stearic acid are crystallized. The pulp is then pumped to a filter press, where the crystals are removed, essentially removing the vast majority of saturated fats from the oil. Depending on the amount of saturated fats in the oil, approximately 5-10% of the oil is lost during this process.

7. Bleaching

The oil is then removed to a bleaching tank at 60° C., where bleaching earth or bentonite earth is added to the oil. Any water in the oil evaporates due to the temperature. Any remaining impurities (trace minerals, etc.) in the oil attach to the bentonite earth. The oil is then run through a bentonite earth filter to remove the bentonite earth together with the impurities.

8. Deodorization

Although not a necessary step, it is advantageous to move the oil to a deodorization tank. The tank contains low vacuum at 120° C. Steam is added at the bottom of the tank, which connects to color and odor molecules (oxidated matter, peroxides) which again travel into the vacuum system and into a residue container. This process gives the oil a neutral color with virtually zero taste and odor.

9. Mixing

The oil is then moved to a separate storage tank. Depending on the concentration of EPA and DHA desired, various batches can be mixed to yield the concentration desired for the final product.

10. Addition of Antioxidants

Antioxidants, in particular mixed tocopherols can be added to the final oil to dramatically reduce the oxidation process. In some embodiments, the antioxidants include rosemary, vitamin E, astaxanthine, carnitine, ascorbyl palmitate, tocopherols or other antioxidants known in the art for omega-3 fatty acids, or derivatives thereof.

11. Drumming

The oil is then drummed in stainless steel drums for storage and topped off with nitrogen to remove oxygen and minimize the potential for oxidation.

12. Flavoring, Coloring and Sweetening Agents

Flavoring agents can also be added to the final oil, either before or after drumming. Useful flavor agents include natural and synthetic flavoring sources including, but not limited to, volatile oils, synthetic flavor oils, flavoring aromatics, oils, liquids, oleoresins and extracts derived from plants, leaves, flowers, fruits, stems and combinations thereof. Useful flavor agents include, citric oils (e.g., lemon, orange, grape, lime and grapefruit) fruit essences (e.g., apple, pear, peach, banana, grape, berry, strawberry, raspberry, blueberry, blackberry, cherry, plum, pineapple, apricot), and/or other fruit flavors. Other useful flavor agents include, e.g., aldehydes and esters (e.g., benzaldehyde (cherry, almond)), citral, i.e., alpha-citral (lemon, lime), neral, i.e., beta-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanal (green fruit), 2-dodedenal (citrus, mandarin) and mixtures thereof, chocolate, cocoa, almond, cashew, macadamia nut, coconut, mint, chili pepper, pepper, cinnamon, vanilla, tooty fruity, mango and green tea. Mixtures of two or more flavor agents may also be employed. When a flavor agent is used, the amount employed will depend upon the particular flavor agent used.

Useful color agents include, e.g., food, drug and cosmetic (FD&C) colors including, e.g., dyes, lakes, and certain natural and derived colorants. Useful lakes include dyes absorbed on aluminum hydroxide and other suitable carriers. Mixtures of color agents may also be employed. When a color agent is employed, the amount used will depend upon the particular color agent used.

Natural and/or artificial sweetening agents can also be added to the composition. Examples of sweeteners include sugars such as sucrose, glucose, invert sugar, fructose, and mixtures thereof, saccharin and its various salts (e.g., sodium and calcium salt of saccharin), cyclamic acid and its various salts, dipeptide sweeteners (e.g., aspartame), dihydrochalcone, and sugar alcohols including, e.g., sorbitol, sorbitol syrup, mannitol and xylitol, and combinations thereof. Natural sweeteners that can be employed include, but are not limited to, luo han, stevia or mixtures thereof. Luo han sweetener is derived from luo han guo fruit (siraitia grosvenorii) that is mainly found in China. It is about 300 times sweeter by weight than sucrose. Luo han is commercially available from, e.g., Barrington Nutritionals (Harrison, N.Y.). Stevia is derived from a South American herb, Stevia rebaudiana. It can be up to about 300 times sweeter than sucrose. Because luo han and stevia have such a sweet taste, only a small amount need be used in the composition. When a sweetening agent is employed the amount used will depend upon the particular sweetening agent used. However, in general, the sweetening agent can constitute from about 0.0005% to about 30%, by weight of the composition. When a sweetener having a very sweet taste, such as luo han or stevia, is used, small amounts such as about 0.0005% to about 0.1% (for example about 0.005% to about 0.015% or about 0.002% to about 0.003%) by weight can be used.

Compositions provided herein can contain additional ingredients. Examples of such additional ingredients include, but are not limited to, vitamins, minerals and/or herbs. As used herein, “vitamin” refers to trace organic substances that are required in the diet, and includes without limitation: thiamin, riboflavin, nicotinic acid, pantothenic acid, pyridoxine, biotin, folic acid, vitamin B 12, lipoic acid, ascorbic acid, vitamin A, vitamin D, vitamin E and vitamin K. Also included within the term vitamin are the coenzymes thereof. Coenzymes are specific chemical forms of vitamins. Coenzymes include thiamine pyrophosphates (TPP), flavin mononucleotide (FMM), flavin adenine dinucleotive (FAD), Nicotinamide adenine dinucleotide (AND), Nicotinamide adenine dinucleotide phosphate (NADP), Coenzyme A (CoA), Coenzyme Q10 (CoQ10), pyridoxal phosphate, biocytin, tetrahydrofolic acid, coenzyme B 12, lipoyllysine, 11-cis-retinal, and 1,25-dihydroxycholecalciferol. The term vitamin(s) also includes choline, camitine, and alpha, beta, and gamma carotenes.

As used herein, “mineral” refers to inorganic substances, metals, and the like required in the human diet. Thus, the tei “mineral” as used herein includes, without limitation, calcium, iron, zinc, selenium, copper, iodine, magnesium, phosphorus, chromium and the like, and mixtures thereof. Compounds containing these elements are also included in the term “mineral.”

As used herein, the term “herb” refers to organic substances defined as any of various often aromatic plants used especially in medicine or as seasoning. Thus, the term “herb” as used herein includes, but is not limited to, black currant, ginsing, ginko bilboa, cinnamon, and the like, and mixtures thereof.

Sources of the omega-3 polyunsaturated fatty acids or derivatives thereof include natural sources including, but not limited to, fish oil (e.g., cod liver oil), flax seed oil, marine oils, sea oils, krill oil, algae and the like. Fish oil is a preferred source.

It is preferred to use a high quality source of omega-3 polyunsaturated fatty acid triglycerides which is rich in omega-3 oils, preferably containing at least 70% omega-3 oils in triglyceride form. The oil may be rich in EPA and may contain DHA. In some embodiments, at least 75% of the omega oils are EPA+DHA. In other embodiments, at least 85% or more are EPA+DHA, with the majority being EPA.

The form in which a composition provided herein is orally administered to the subject is not critical. In some embodiments, the composition is administered as a liquid, as a dispersion or in a capsule. In some embodiments, the composition is administered in the form of individual doses. In certain embodiments, the composition is administered to the patient enterally as a liquid or dispersion, e.g., via a feeding tube.

In some embodiments, a composition provided herein is administered in the form of a daily dose. However, depending on the severity of the condition being treated, this may not be required, and the period between administration of the doses may be longer than one day. In addition, the term “administer” includes both the case where a third party administers the dose to the subject and the case where the subject self-administers the dose.

### EXAMPLES

### Example 1

### EPA/DHA Formulations

The following table shows an example of a liquid formulation which may be used in the methods provided herein:

| Component (per 5 ml/4555 mg serving) | Amount (mg) |
|---|---|
| Omega-3 fatty acid content | 2733 |
| EPA* | 1366 |
| DHA* | 911 |
| Other omega-3 fatty acids | 456 |
| Omega-6 fatty acid content | 180 |
| EPA:DHA (wt. ratio) | ~1.5:1 |

*triglyceride form (~90%)

### Example 2

### Treatment of Traumatic Brain Injury With EPA/DHA Mixture

Here we present a case that was intentionally treated with substantial amounts of omega-3 fatty acids (n-3FA) to provide the nutritional foundation for the brain to begin the healing process following severe TBI. A teenager sustained a severe TBI in a motor vehicle accident. After prolonged extrication, he was resuscitated at the scene and flown to a Level I Trauma Center. H is Glasgow Coma Scale score was three. Computerized tomography (CT) revealed panhemispheric right subdural and small temporal epidural hematomas and a three millimeter midline shift. The patient underwent emergency craniotomy and ICP monitor placement. The patient was rated at Rancho Los Amigos Cognitive Scale Level I and the attending neurosurgeon's impression was that the injury was likely lethal.

On hospital day ten, magnetic resonance imaging revealed a right cerebral convexity subdural hemorrhage and abnormal FLAIR signals consistent with diffuse axonal injury. Believed to be in a peimanent vegetative state, a tracheotomy and percutaneous endoscopic gastrostomy (PEG) tube were placed for custodial care and enteral feedings were started (Promote; 80 ml/hour; 1920 kcal per day). The following day, omega-3 fatty acids were added to enteral feedings. The patient began receiving Ultimate Omega® n-3FA supplement at a dose of 15 ml twice a day (30 ml/day), providing 9,756 mg EPA, 6,756 mg DHA, and 19,212 mg total n-3FA daily via his PEG.

On day 21, the patient was weaned off the ventilator and transported to a specialized rehabilitation institute three days later. H is level of functioning was measured at Rancho Los Amigos Level III. The patient began therapy, which gradually led to cognitive and physical improvements. He was discharged to home four months after the injury.

Over the following year, the patient remained on omega-3 fatty acids using ProOmega-D supplement (the professional version of Ultimate Omega®) which also provided Vitamin D3 (6000 International Units). The patient remained on this level of omega-3 fatty acids for over one year and experienced no side effects. Two years later, the patient is at Rancho Los Amigos Level VIII, but has speech and balance issues consistent with the location and size of the brain damage, and is walking with the aid of a cane due to significant left sided weakness.

All of the references cited herein are incorporated by reference in their entirety. While the methods provided herein have been described with respect to the particular embodiments, it will be apparent to those skilled in the art that various changes and modifications can be made without departing from the spirit and scope as recited by the appended claims.

The embodiments described above are intended to be merely exemplary and those skilled in the art will recognize or will be able to ascertain using no more than routine experimentation, numerous equivalents of specific compounds, materials and procedures. All such equivalents are considered to be within the scope and are encompassed by the appended claims.

What is claimed is:

1. A method of treating a brain injury in a patient, the method comprising oral administration of a composition comprising an omega-3 fatty acid mixture to a patient having a brain injury, wherein the omega-3 fatty acid mixture comprises at least about 70% by weight of a combination of (a) EPA glyceride, and (b) DHA glyceride, in a weight ratio of a:b of from about 1.5:1 to about 1.3:1.

2. The method of claim 1, wherein the omega-3 fatty acid mixture comprises at least about 80% by weight of a combination of (a) EPA glyceride, and (b) DHA glyceride, in a weight ratio of a:b of from about 1.5:1 to about 1.3:1.

3. The method of claim 1, wherein the omega-3 fatty acid mixture comprises at least about 85% by weight of a combination of (a) EPA glyceride, and (b) DHA glyceride, in a weight ratio of a:b of from about 1.5:1 to about 1.3:1.

4. The method of claim 1, wherein the ratio of a:b is from about 1.5:1 to about 1.4:1.

5. The method of claim 4, wherein the ratio of a:b is about 1.4:1.

6. The method of claim 4, wherein the ratio of a:b is about 1.5:1.

7. The method of claim 1, wherein the triglyceride content of each of the EPA glyceride and the DHA glyceride is about 80% by weight.

8. The method of claim 1, wherein the triglyceride content of each of the EPA glyceride and the DHA glyceride is about 85% by weight.

9. The method of claim 1, wherein the triglyceride content of each of the EPA glyceride and the DHA glyceride is about 90% by weight.

10. The method of claim 1, wherein the triglyceride content of each of the EPA glyceride and the DHA glyceride is about 95% by weight.

11. The method of claim 1, wherein the triglyceride content of each of the EPA glyceride and the DHA glyceride is about 99% by weight.

12. The method of claim 1, wherein about 15,000 mg to about 25,000 mg of the combination of (a) and (b) is administered daily.

13. The method of claim 1, wherein about 15,000 mg to about 20,000 mg of the combination of (a) and (b) is administered daily.

14. The method of claim 1, wherein about 15,000 mg to about 16,000 mg of the combination of (a) and (b) is administered daily.

15. The method of claim of claim 1, wherein about 7,500 mg to about 10,000 mg of the combination of (a) and (b) is administered twice daily.

16. The method of claim of claim 1, wherein about 7,500 mg to about 8,000 mg of the combination of (a) and (b) is administered twice daily.

17. The method of claim 1, wherein the composition is a liquid foiniulation.

18. The method of claim 1, wherein the brain injury is a traumatic brain injury.

19. The method of claim 18, wherein the treatment prevents damage to the brain associated with a traumatic brain injury by reducing secondary brain trauma in the patient post-acute brain injury.

20. The method of claim 1, wherein the brain injury is an anoxic brain injury.
